(19) United States
(12) Patent Application Publication
(10) Pub. No.: US 2023/0082404 A1
(43) Pub. Date: Mar. 16, 2023

(54) CLINICAL ROTATION MANAGEMENT SYSTEM

(71) Applicant: Sal Rehman, Irvine, CA (US)

(72) Inventor: Sal Rehman, Irvine, CA (US)

(21) Appl. No.: 17/474,498

(22) Filed: Sep. 14, 2021

(51) Int. Cl. G16H 40/20; G06Q 10/06

(52) U.S. Cl. CPC G16H 40/20; G06Q 10/06315; G06Q 10/06311

(57) ABSTRACT

A clinical rotation management system is disclosed, comprising a healthcare professional management module to determine the availability of a healthcare professional to oversee a clinical rotation of a student. An education management module receives a plurality of educational credentials from a student management module and determines if the student is eligible for the clinical rotation. A clinical rotation management module schedules the clinical rotation for a student and corresponds the clinical rotation to the healthcare professional. A learning management system includes a training generation engine to permit the healthcare professional to create a training implement, wherein the training implement is provided to a student device to permit the student to interact with the training implement.

### TECHNICAL FIELD

The embodiments generally relate to computerized systems for the management of clinical rotations and training for students.

### BACKGROUND

Physicians, physician assistants, nurses, and other medical professionals often spend many years in school learning the theories, techniques, and base knowledge of their area of practice. Many of these professionals are required to spend a portion of their time in a hospital or other clinical setting, known as “clinical rotations.” These clinical rotations are often required by state law in order for the practitioner to receive their license and/or by the medical, physician assistant, or nursing school to qualify for graduation.

Scheduling clinical rotations has historically been the sole responsibility of the student. This can be frustrating as each student must contact clinical sites and medical professionals thereof to schedule a clinical rotation. Once scheduled, the student must then find temporary housing, complete necessary training, receive any required immunizations, obtain a visa (typically needed for foreign students), among other hurdles. Each clinical rotation may only last a few weeks or months, requiring the student to undergo each step of scheduling their clinical rotations for multiple clinical sites. Further, each clinical site may require specific training related to the specific facility, area of practice, etc., which may take considerable time at the beginning of the clinical rotation to complete.

In the current arts, some systems have been created which serve as a form of client relationship management (CRM) system to help supervising practitioners manage their students who are undergoing rotations. While useful for the practitioners, this does not aid the students in finding and scheduling clinical rotations. Further, these systems do not include integrated training systems provided during the preparation period before or during a clinical rotation.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The embodiments provided herein relate to a clinical rotation management system, the system comprising a healthcare professional management module to determine the availability of a healthcare professional to oversee a clinical rotation of a student. An education management module receives a plurality of educational credentials from a student management module and determines if the student is eligible for the clinical rotation. A clinical rotation management module schedules the clinical rotation for a student and corresponds the clinical rotation to the healthcare professional. A learning management system includes a training generation engine to permit the healthcare professional to create a training implement, wherein the training implement is provided to a student device to permit the student to interact with the training implement.

In one aspect, a training module permits the student to interact with the training implement.

In one aspect, a status indicator indicates the student's progress through the training implement.

In one aspect, the training examination engine includes at least one of the following: an audio processor, a video processor, and a text processor.

In one aspect, an examination administration engine administers the examination to the student.

In one aspect, a communications engine permits the student and the healthcare professional to communicate via the student device and a healthcare professional device.

In one aspect, a student database stores a plurality of student information, a healthcare professional database stores a plurality of healthcare professional information, and a clinical site database stores a plurality of clinical site information.

In one aspect, information from the student database, the healthcare professional database, and the clinical site database are associated with a user profile and displayed on the user profile.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a block diagram of the network infrastructure, according to some embodiments;

FIG. 2 illustrates a block diagram of the application system, according to some embodiments;

FIG. 3 illustrates a block diagram of the clinical system infrastructure, according to some embodiments;

FIG. 4 illustrates a block diagram of the clinical rotation management system, according to some embodiments;

FIG. 5 illustrates a block diagram of the scheduling engine, according to some embodiments;

FIG. 6 illustrates a block diagram of the communications engine, according to some embodiments;

FIG. 7 illustrates a screenshot of the medical professional account interface, according to some embodiments;

FIG. 8 illustrates a screenshot of the student account interface, according to some embodiments;

FIG. 9 illustrates a screenshot of the clinical facility interface, according to some embodiments;

FIG. 10 illustrates a screenshot of the navigational tabs interface, according to some embodiments;

FIG. 11 illustrates a block diagram of the learning management system, according to some embodiments;

FIG. 12 illustrates a block diagram of the training generation engine, according to some embodiments;

FIG. 13 illustrates a block diagram of the examination administration engine, according to some embodiments; and

FIG. 14 illustrates a block diagram of the training module, according to some embodiments.

### DETAILED DESCRIPTION

The specific details of the single embodiment or variety of embodiments described herein are to the described system and methods of use. Any specific details of the embodiments are used for demonstration purposes only, and no unnecessary limitations or inferences are to be understood therefrom.

Before describing in detail exemplary embodiments, it is noted that the embodiments reside primarily in combinations of components and procedures related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided herein relate to a clinical rotation management system, which connects students with medical professionals during the process of finding and undergoing a clinical rotation. The system can be utilized with outpatient and inpatient clinical sites to schedule and manage the various communications, training, and processes involved in gaining hands-on experience during a clinical rotation.

As used herein, the term “user” may relate to a student or a medical professional unless further specified by the terms “student” or “medical professional.”

As used herein, the term “student” relates to a user of the system who is a student in a medical professional educational program (e.g., medical school, nursing school, physician's assistant school, etc.).

As used herein, the term “medical professional” relates to any medical professional or professional involved in medical staffing and scheduling within a healthcare facility (physician, nurse, supervisor, administrator, etc.).

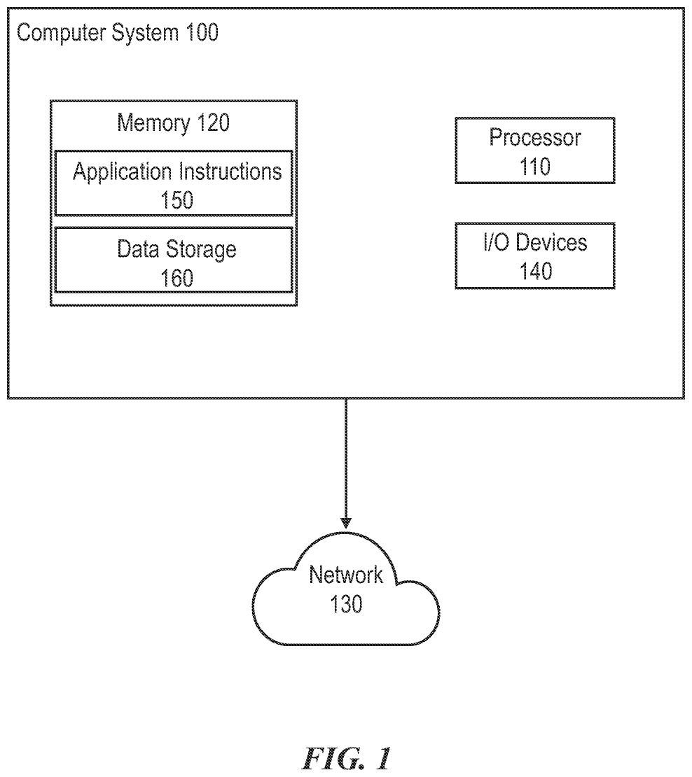

FIG. 1 illustrates a computer system 100, which may be utilized to execute the processes described herein. The computer system 100 is comprised of a standalone computer or mobile computing device, a mainframe computer system, a workstation, a network computer, a desktop computer, a laptop, or the like. The computer system 100 includes one or more processors 110 coupled to a memory 120 via an input/output (I/O) interface. Computer system 100 may further include a network interface to communicate with the network 130. One or more input/output (I/O) devices 140, such as video device(s) (e.g., a camera), audio device(s), and display(s) are in operable communication with the computer system 100. In some embodiments, similar I/O devices 140 may be separate from computer system 100 and may interact with one or more nodes of the computer system 100 through a wired or wireless connection, such as over a network interface.

Processors 110 suitable for the execution of a computer program include both general and special purpose microprocessors and any one or more processors of any digital computing device. The processor 110 will receive instructions and data from a read-only memory or a random-access memory or both. The essential elements of a computing device are a processor for performing actions in accordance with instructions and one or more memory devices for storing instructions and data. Generally, a computing device will also include, or be operatively coupled to receive data from or transfer data to, or both, one or more mass storage devices for storing data, e.g., magnetic, magneto-optical disks, or optical disks; however, a computing device need not have such devices. Moreover, a computing device can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

A network interface may be configured to allow data to be exchanged between the computer system 100 and other devices attached to a network 130, such as other computer systems, or between nodes of the computer system 100. In various embodiments, the network interface may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol.

The memory 120 may include application instructions 150, configured to implement certain embodiments described herein, and a database 160, comprising various data accessible by the application instructions 150. In one embodiment, the application instructions 150 may include software elements corresponding to one or more of the various embodiments described herein. For example, application instructions 150 may be implemented in various embodiments using any desired programming language, scripting language, or combination of programming languages and/or scripting languages (e.g., C, C++, C#, JAVA®, JAVASCRIPT®, PERL®, etc.).

The memory may be utilized to store user information including physician information (name, contact information, associated clinical sites, national provider identifier (NPI), etc.), student information (name, contact information, associated educational institution, immunization information, certifications, etc.), clinical rotation site information, and the like.

The steps and actions of the computer system 100 described in connection with the embodiments disclosed herein may be embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

Also, any connection may be associated with a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. “Disk” and “disc,” as used herein, include compact disc (CD), laser disc, optical disc, digital versatile disc (DVD), floppy disk and Blu-ray disc where disks usually reproduce data magnetically, while discs usually reproduce data optically with lasers. Combinations of the above should also be included within the scope of computer-readable media.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

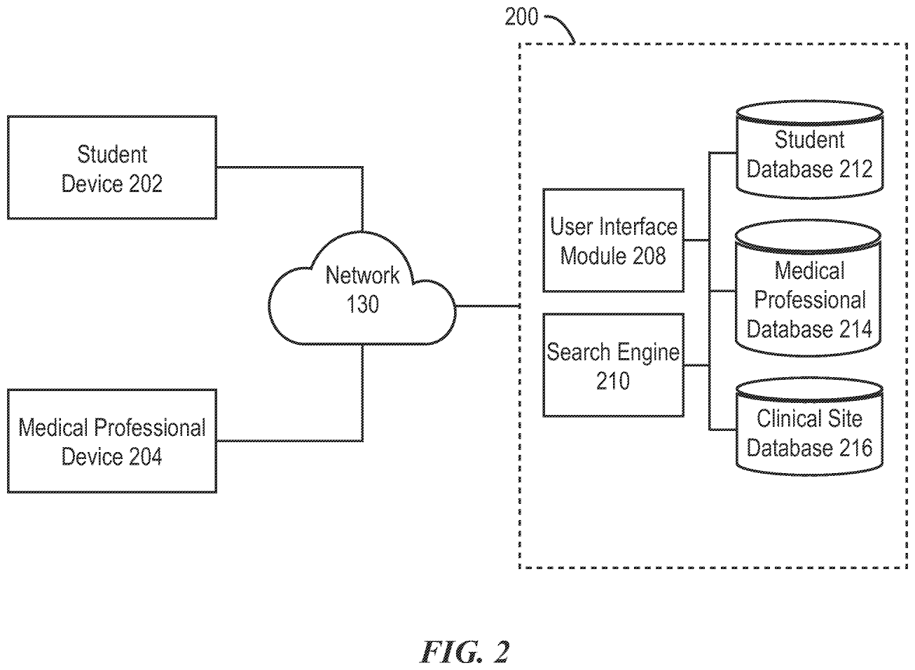

FIG. 2 illustrates a block diagram of the application system 200 in operable communication with a student device 202 and a medical professional device 204 via a network 130. The application system 200 includes a user interface module 208 and search engine 210 to search information and display information received from a student database 212, medical professional database 214, and clinical site database 216. The student database 212 stores student information input by the student to a user profile. The student information may be utilized by the medical professional to determine if the candidate is suitable for the clinical rotation, has completed necessary training and education, has the correct immunizations, has a valid visa, etc. The medical professional database 214 includes information associated with the medical professionals utilizing the system. The students may search medical professionals to determine which medical professionals they would like to contact and communicate with. The clinical site database 216 stores clinical site information include the required training, immunizations, site location, etc.

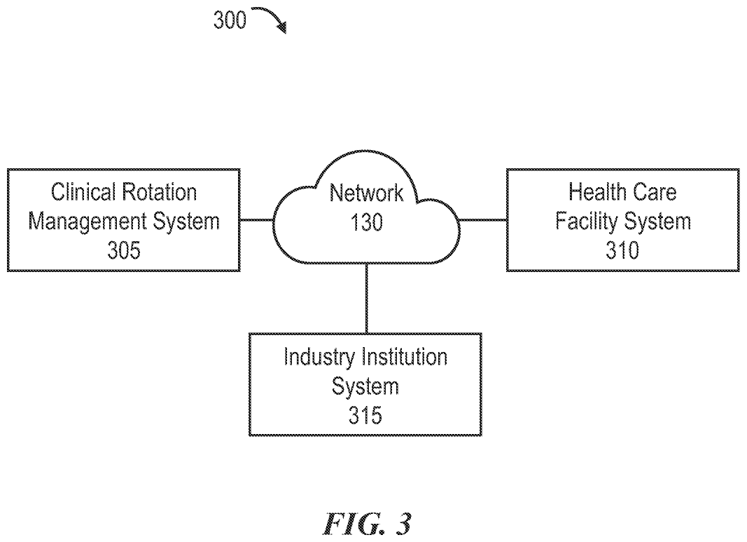

FIG. 3 illustrates a block diagram of the clinical system infrastructure 300 which may include a clinical rotation management system 305, a healthcare facility system 310, and an educational institution system 315 each in communication with one another via network 130. One skilled in the arts will readily appreciate that each of the clinical rotation management system 305, healthcare facility system 310, and educational institution system 315 may not necessarily be a singularity (provided in FIG. 3 for illustrative purposes only), but may include a plurality of one or more of the clinical rotation management system 305, healthcare facility system 310, and educational institution system 315.

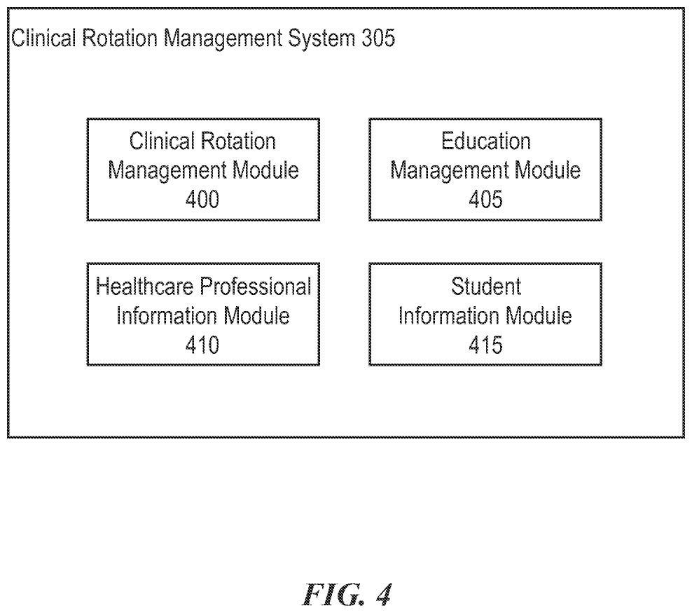

FIG. 4 illustrates a block diagram of the clinical rotation management system 305, which may include a clinical rotation management module 400, and education management module 405, a healthcare professional information module 410, and a student information module 415. Each module may include any suitable form of hardware, software, firmware, and/or combination of hardware, software, and firmware. For example, the modules may be created by a computer executing a code stored on a computer readable medium, such as a computer memory.

The clinical rotation management module 400 is operable to determine a suitable clinical rotation based on user inputs including available date ranges, preferred locations, availability of the supervising medical professional, and the like. The clinical rotation management module 400 may determine if the student is eligible for one or more of the various available clinical rotations provided on the user interface. For example, the clinical rotation management module 400 may determine if the student has their required training, educational background, and immunizations, to qualify at a particular healthcare facility.

The education management module 405 receives educational information including the students' progress, credentials, certifications, and the like. In such, the education management module 405 communicates with the clinical rotation management module 400 to determine if the student is eligible for the clinical rotation, and at which stage of clinical rotation the student is at.

In some embodiments, the education information module 405 may be configured to receive and manage information related to clinical rotations from one or more schools, such as a medical school, a physician's assistant (“PA”) school, a nursing school, or other caregiver educational school or institution. For example, the school information module 405 may be configured to store the school information in the clinical site database 216. The stored information may then be employed by the clinical rotation management module 400 to create and manage clinical rotation schedules. Accordingly, the school information module 405 may receive and manage any one of a number of suitable types of clinical rotation related data. For example, the module 405 may receive student information, faculty information, course information, affiliation information, and/or preference information.

The healthcare professional information module 410 will receive information pertaining to each healthcare professional to actively display up-to-date information of the healthcare professional's availability, specialties, location, etc. The student information module 415 receives student information and will display up-to-date information related to availability, experience, clinical rotation history, etc., such that it may be viewed by healthcare professionals.

In some embodiments, healthcare professional information may include, but is not limited to, faculty names, titles, offices, addresses, contact information (e.g., phone numbers, email addresses), degrees, medical certifications, courses taught, course schedules, photographs, qualifications (e.g., background checks, CPR certification, drug screening, immunization records, security clearance, and the like), schedules, assigned rotations, faculty status, and so forth. Course information may include course names, course type (e.g., clinical rotation or lecture) semester/year taught, course number, course description, usual faculty, assigned faculty, course duration, course size, rotation group information, course site, start times, end times, pre-rotation and post-rotation times for students and faculty, and so forth.

In some embodiments, the student information may include, but is not limited to a student's name, ID number, gender, contact information (e.g., address, telephone numbers, email addresses), semester/year in school, background check status, qualifications (e.g., CPR certification, drug screening, immunizations, security clearance, and so forth), class schedules, addresses, grades, course numbers and schedules, health records, currently scheduled clinical rotations, desired clinical rotations, emergency contact information, special accommodations, travel restrictions (e.g., maximum travel time), workday preferences, and the like.

In some embodiments, the clinical rotation management module 400 may be configured to enable users to request the creation of new clinical rotations. For example, in one embodiment, the clinical rotation management module 400 may be configured to generate a data input screen that enables a school administrator to request approval for a new clinical rotation from a medical facility, such as a hospital, clinic, or doctor's office. The clinical rotation management module 400 may also be configured to create clinical rotation schedules and to enable users to view/edit the created clinical rotation schedules.

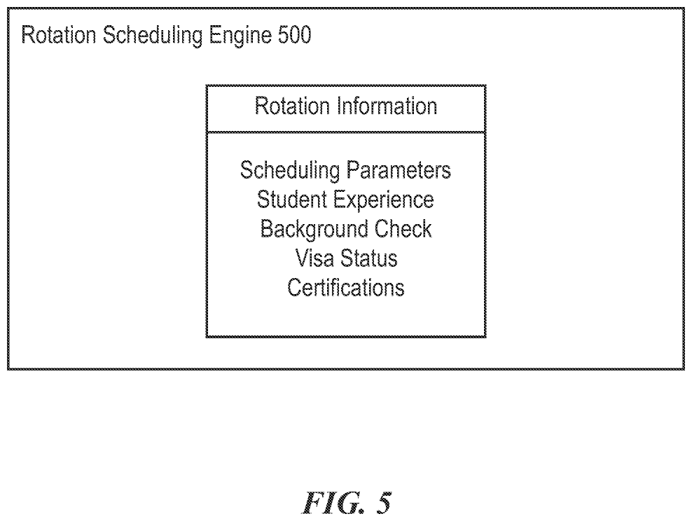

FIG. 5 illustrates a rotation scheduling engine 500 wherein the system receives input from the student and/or the healthcare professional related to a plurality of rotation parameters. The rotation parameters may include, but are not limited to, background check guidelines, drug screening guidelines, immunization guidelines, security guidelines, certification parameters (e.g., CPR certification, course work, etc.), supervisor to student ratios, and so forth. Other information may include details, such as starting and ending dates, for affiliation agreements between the medical facility and any schools that are affiliated therewith.

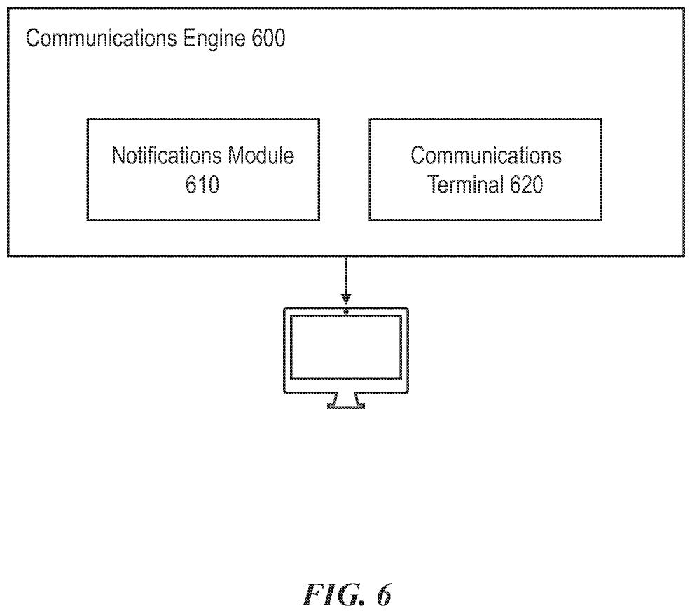

FIG. 6 illustrates a block diagram of the communications engine 600, which may include a notifications module 610 to provide notifications to the student and/or medical professional upon a new clinical rotation, updates regarding the clinical rotation, scheduling changes, location changes, status updates, and the like. A communications terminal 620 allows any user of the system to transmit communication via text, audio, video, etc. to other users within the system. For example, the communications terminal 620 may allow the student to request an interview with a medical professional, which is then conducted using the system described herein.

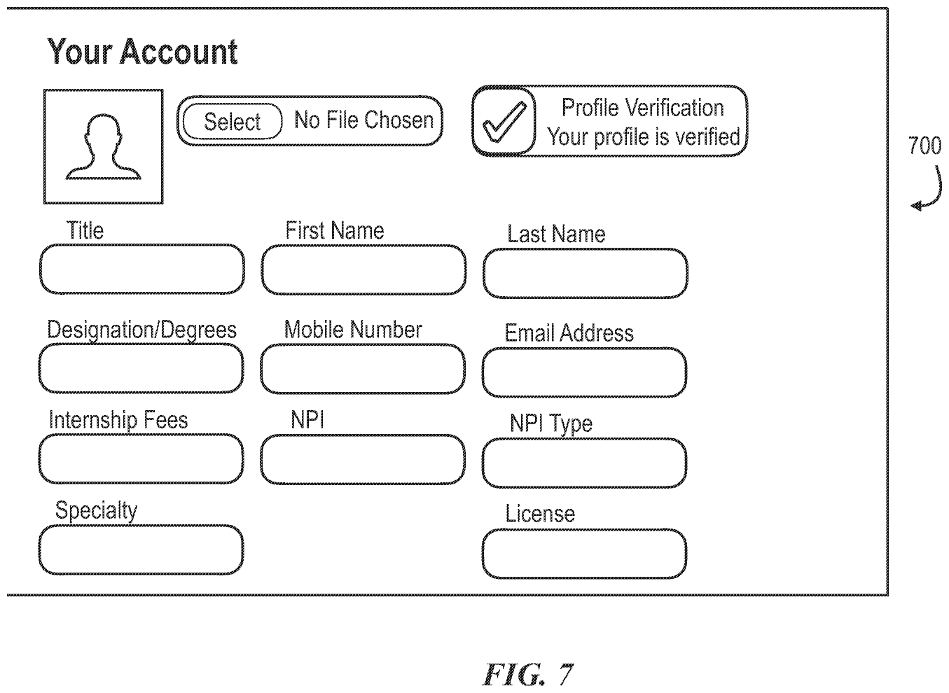

FIG. 7 illustrates a screenshot of the medical professional account interface 700 comprising a plurality of medical professional information related to a medical professional, which is displayed on the medical professional profile. The medical professional information may include the title, name, designation and degrees, contact information, internship fees, NPI number, NPI type, specialty, and medical license information, among other information known in the arts. The medical professional may select to upload an image. One skilled in the arts will readily understand that various additional account features may be provided such as managing payments, account details and preferences, etc.

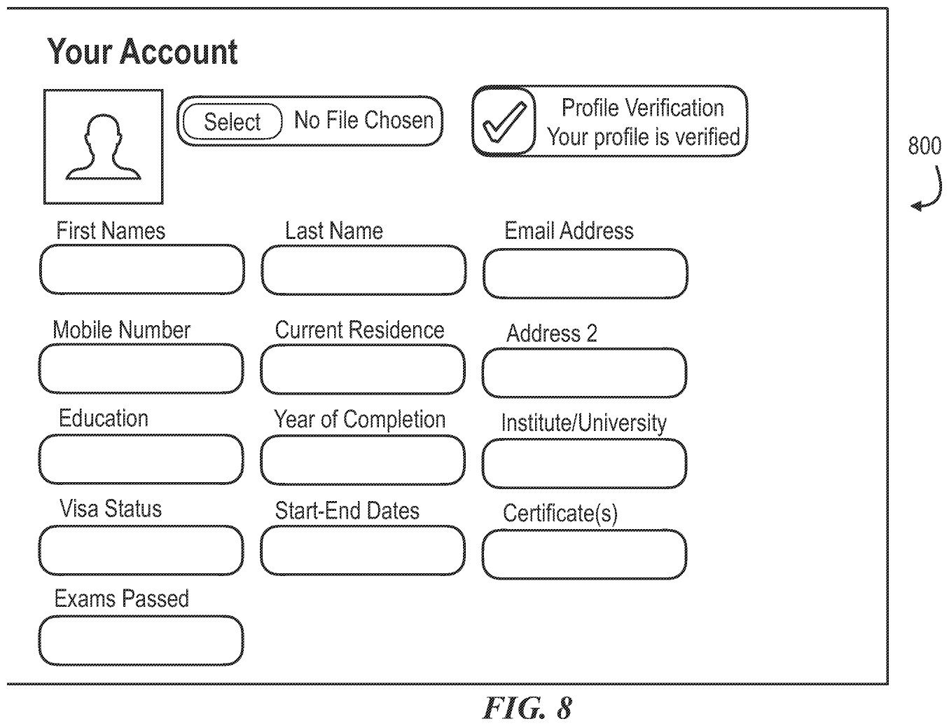

FIG. 8 illustrates a screenshot of the student account interface 800 including a plurality of student information. The student information may include names, contact information, education history, education status, name of the university or institute, visa status, certifications, preferred start and/or end dates, and examination history. In such the student information will be uploaded to the system and transmitted to healthcare professionals, medical facilities, and/or educational facilities when required.

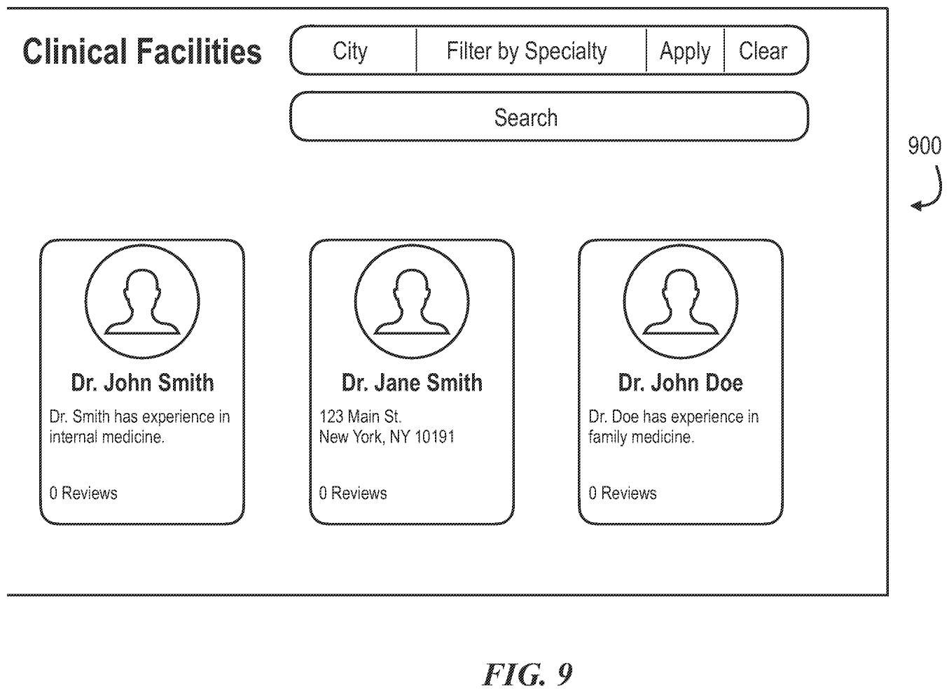

FIG. 9 illustrates a screenshot of the clinical facilities interface 900 wherein the student (or other user) may search for clinical facilities at which they wish to undergo a clinical rotation. The user may search for clinical facilities by location, specialty, name, and other search parameters known in the arts. The student will then be displayed a listing of clinical facilities and/or healthcare professionals at the matching clinical facilities who are signed up with the system and meet the search parameters. For example, a plurality of healthcare professionals who meet the students preferred start date and specialty are displayed. The student may then view the healthcare professional's profile by selecting the tab corresponding to the healthcare professional. The student may then be prompted to submit an application, engage in communication with the healthcare professional, schedule a meeting, etc.

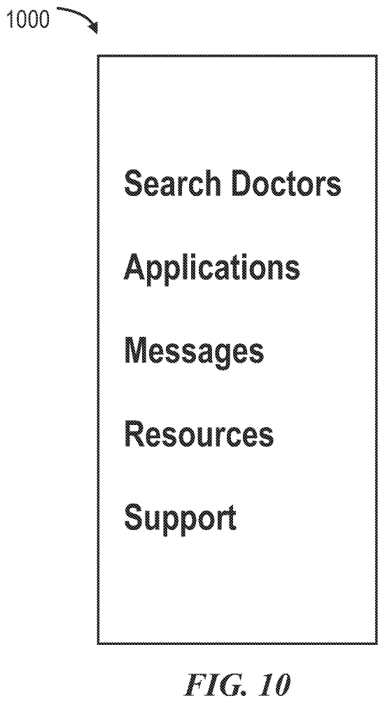

FIG. 10 illustrates a screenshot of the navigation tabs 1000 including a “search doctors tab”, “applications tab”, “messages tab”, “resources tab”, and “support tab”. The search doctors tab provides the interface described on FIG. 9, wherein the student is able to search doctors and/or medical facilities based on user-input search parameters. The applications tab allows the student to submit applications to medical professionals, healthcare facilities, and the like, as well as to manage applications that have previously been submitted. The messages tab allows the student to communicate with other users, including medical professionals, education professionals, other students, training personnel etc. The resources tab provides the user with various resources required to interact with the system described hereinabove, resources provided by the healthcare facilities and/or medical professionals, and the like.

The system further includes a means for providing training to students prior to or during their clinical rotations. For example, each clinical rotation may be executed at a different clinical site within different geographical locations. Each clinical rotation site, state or local laws, regulatory bodies, and other guidelines may require specific safety training, operational procedure training, or site-specific training. It is a goal of the system to provide this training, which can be at least partially completed via the student's computing device.

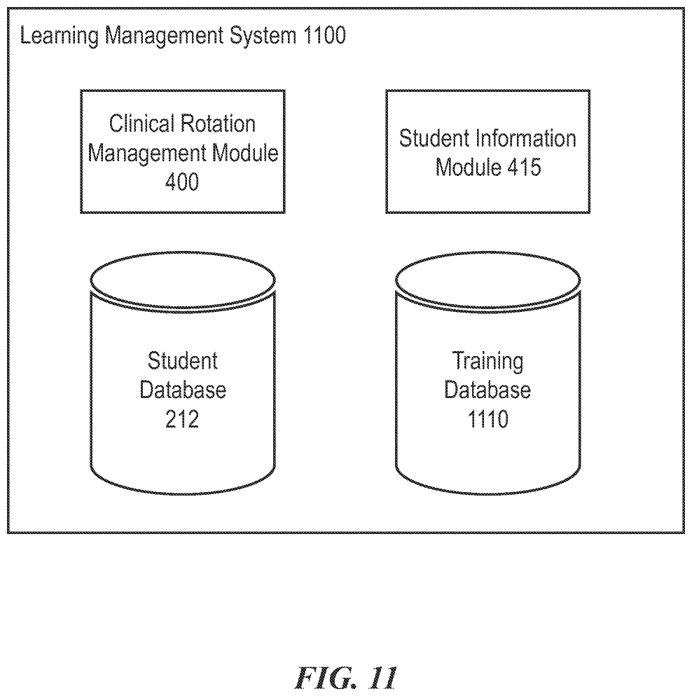

FIG. 11 illustrates a block diagram of the learning management system 1100 which may be utilized by the clinical rotation management module 400 to ensure completion of required training before or during a clinical rotation. The learning management system 1100 may also communicate completed training credentials to the student information module 415 and student database 212 such that training credentials are automatically saved to the student database and applied by the student information module 415 to the student's credentials. A training database 1110 may store training information, which may be searched, displayed, and interacted with by the student.

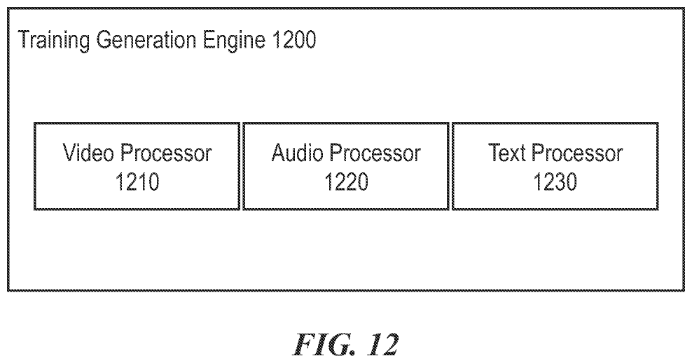

FIG. 12 illustrates a block diagram of the training generation engine 1200, which may be used by the medical professional, healthcare facility, and/or administrative users to create training implements to be used by the students. The training generation engine 1200 may include a video processor 1210, audio processor 1220, and text processor 1230 to permit the user to input information related to a training topic. For example, the healthcare facility may create training videos providing a virtual tour of the medical facility using the training generation engine 1200 and video processor 1210. In another example, the medical professional may create a video detailing an operational procedure common to their practice. The medical professional may then submit the video to the training database 1110.

In some embodiments, the creator of the training implement may indicate checkpoints along the course of the training, which provide the student the opportunity to review information, focus on particularly important material, etc. Further, the creator may provide a supplemental examination (i.e., a quiz) at various points throughout the training.

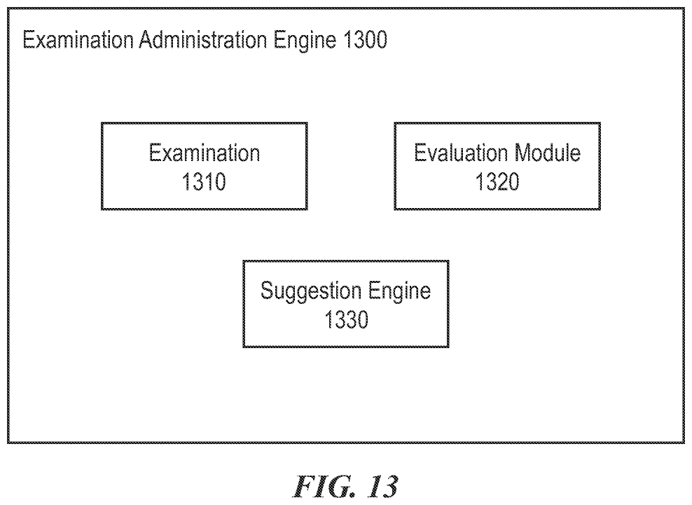

FIG. 13 illustrates a block diagram of the examination administration engine 1300, which may administer an examination 1310 to the student. The examination 1310 may correspond to a training segment, previous experience, be used as a diagnostic examination, as a post-rotation examination, and the like. The examination may be administered as a written examination, oral examination, or other form of examination known in the arts. Once complete, the examination 1310 may be automatically evaluated by an evaluation module 1320 to assign a grade to the examination 1310. The suggestion engine 1330 may transmit suggested materials, suggested answers, etc. to the student for questions provided during the examination. For example, the suggestion module may suggest the correct answer and a corresponding training exercise, which has taught the corresponding information.

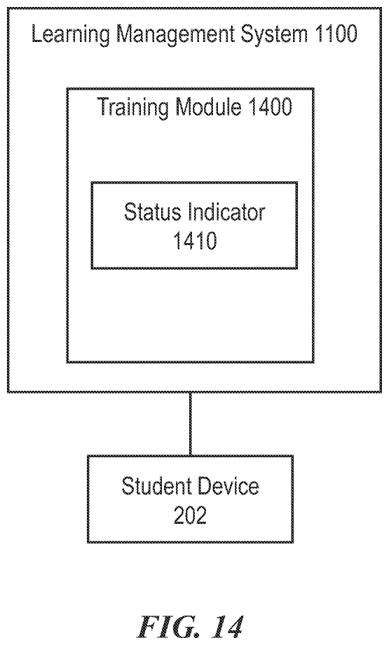

FIG. 14 illustrates a block diagram of the training module 1400 in operable communication with the learning management system 1100. The training module 1400 allows the student to interact, via the student device 202, with various training exercises provided by the medical facility, medical professional, or administrative user. The training module 1400 may provide a status indicator 1410 to indicate the status of the students' progress through a training exercise or series of training exercises. Alternatively, the user may be provided with a status indicator 1410 of whether or not their completed training is accepted by a particular medical facility, medical professional, etc. In one example, the student may select one or more training videos to watch and interact with (if interactive capabilities are provided) in order to complete the training. If the student completes the training, their profile may be updated with the certificate of completion, and/or a notification may be sent to the healthcare facility, healthcare professional, or other associated user or group thereof.

In some embodiments, the medical professional, healthcare facility, or other user may provide guidelines, informational posts, and the like related to advice to the students. Advice may include information related to how to be a successful clinician, how to find a job, or how to effectively communicate with patients.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A clinical rotation management system, comprising:
a healthcare professional management module to determine the availability of a healthcare professional to oversee a clinical rotation of a student;
an education management module to receive a plurality of educational credentials from a student management module, the education management module to determine if the student is eligible for the clinical rotation;
a clinical rotation management module to schedule the clinical rotation for a student and correspond the clinical rotation to the healthcare professional;
a learning management system comprising a training generation engine to permit the healthcare professional to create a training implement, wherein the training implement is provided to a student device to permit the student to interact with the training implement.

2. The system of claim 1, further comprising a training module to permit the student to interact with the training implement.

3. The system of claim 2, further comprising a status indicator to indicate the students' progress through the training implement.

4. The system of claim 3, wherein the training examination engine includes at least one of the following: an audio processor, a video processor, and a text processor.

5. The system of claim 4, further comprising an examination administration engine to administer the examination to the student.

6. The system of claim 1, further comprising a communications engine to permit the student and the healthcare professional to communicate via the student device and a healthcare professional device.

7. The system of claim 1, further comprising a student database to store a plurality of student information.

8. The system of claim 7, further comprising a healthcare professional database to store a plurality of healthcare professional information.

9. The system of claim 8, further comprising a clinical site database to store a plurality of clinical site information.

10. The system of claim 9, wherein information from the student database, the healthcare professional database, and the clinical site database are associated with a user profile and displayed on the user profile.

11. A clinical rotation management system, comprising:
a healthcare professional management module to determine the availability of a healthcare professional to oversee a clinical rotation of a student;
an education management module to receive a plurality of educational credentials from a student management module, the education management module to determine if the student is eligible for the clinical rotation;
a clinical rotation management module to schedule the clinical rotation for a student and correspond the clinical rotation to the healthcare professional, wherein the clinical rotation management module permits the student to transmit a request for a clinical rotation, wherein the request includes at least one of the following: a start date, an end date, a healthcare professional, a clinical site, and a specialty;
a learning management system comprising a training generation engine to permit the healthcare professional to create a training implement, wherein the training implement is provided to a student device to permit the student to interact with the training implement, wherein the learning management system determines the completion of the training implement to and transmits a completed training implement to the education management module.

12. The system of claim 11, further comprising a training module to permit the student to interact with the training implement.

13. The system of claim 12, wherein the training examination engine includes at least one of the following: an audio processor, a video processor, and a text processor.

14. The system of claim 13, further comprising an examination administration engine to administer the examination to the student.

15. The system of claim 14, further comprising a communications engine to permit the student and the healthcare professional to communicate via the student device and a healthcare professional device.

16. The system of claim 15, further comprising a student database to store a plurality of student information.

17. The system of claim 16, further comprising a healthcare professional database to store a plurality of healthcare professional information.

18. The system of claim 17, further comprising a clinical site database to store a plurality of clinical site information.

19. The system of claim 18, wherein information from the student database, the healthcare professional database, and the clinical site database are associated with a user profile and displayed on the user profile.

20. A clinical rotation management system, comprising:
a healthcare professional management module to determine the availability of a healthcare professional to oversee a clinical rotation of a student;
an education management module to receive a plurality of educational credentials from a student management module, the education management module to determine if the student is eligible for the clinical rotation;
a clinical rotation management module to schedule the clinical rotation for a student and correspond the clinical rotation to the healthcare professional, wherein the clinical rotation management module permits the student to transmit a request for a clinical rotation, wherein the request includes at least one of the following: a start date, an end date, a healthcare professional, a clinical site, and a specialty;
a learning management system comprising a training generation engine to permit the healthcare professional to create a training implement, wherein the training implement is provided to a student device via a training module to permit the student to interact with the training implement, wherein the learning management system determines the completion of the training implement to and transmits a completed training implement to the education management module;
a student profile to display a plurality of student information stored in a student database, wherein the plurality of student profile information comprises at least one of the following: credentials, educational history, training history, clinical rotations, wherein the student profile is viewable by the healthcare professional.
